(19) United States
(12) Patent Application Publication
(10) Pub. No.: US 2010/0196521 A1
(43) Pub. Date: Aug. 5, 2010

(54) Formulation for the treatment of hypoxia and related disorders

(76) Inventor: Yuk Kwan LIU, Tuen Mun (HK)

Correspondence Address: EAGLE IP LIMITED, 13/F, Bright Way Tower, 33 Mong Kok Road, Kowloon, HK

(21) Appl. No.: 12/700,729

(22) Filed: Feb. 5, 2010

Related U.S. Application Data
(60) Provisional application No. 61/150,005, filed on Feb. 5, 2009.

(51) Int. Cl. A61K 36/752; A61K 36/18; A61Q 19/08; A61Q 19/10

(52) U.S. Cl. 424/736; 424/725

(57) ABSTRACT

A plant extract formulation is described comprising lemon oil, eucalyptus oil, basil oil, davana oil, rosewood oil, fennel oil and citronella oil. The formulation is used for the treatment of hypoxia and related disorders.

### CROSS-REFERENCE TO RELATED APPLICATION

This application claims benefit under 35 U.S.C. §119(e) of U.S. Provisional Application having Ser. No. 61/150,005 filed Feb. 5, 2009 which is hereby incorporated by reference herein in its entirety.

### FIELD OF INVENTION

This invention relates to a formulation and in particular a formulation specifically for the treatment of hypoxia and related disorders.

### BACKGROUND OF INVENTION

Modern medical researches often focus on single chemical to narrow the scope of research, and the toxicity and side effects of a single chemical can be very intense. Traditional Chinese medicine utilizes natural animals, plants and minerals as the research objects. However, due to its immense ingredients and complexity, the drug action of traditional Chinese medicine products is slow and thus the effect is often not that significant, often prolonging the overall treatment period.

Hypoxia is a condition in which tissues and/or cells cannot fully utilize oxygen and is caused by malfunctioning or poor oxygen binding ability of red blood cells. Hypoxia will bring different levels of functional injuries and damages to the systems of our body such as the central nervous system, the respiratory system, and the circulation system.

### SUMMARY OF INVENTION

In the light of the foregoing background, it is an object of the present invention to provide an alternative for the treatment of hypoxia and related disorders.

Accordingly, the present invention, in one aspect, is a plant extract formulation comprising a mixture of the following components: lemon oil, eucalyptus oil, basil oil, davana oil, rosewood oil, fennel oil, and citronella oil.

In a preferred embodiment of the present invention, the relative ratio of the various components of the formulation is listed in Table 1, with good efficacy for treatment hypoxia and other related disorders. In another preferred embodiment, the formulation is used for the treatment of hypoxia and related disorders.

In another preferred embodiment, the formulation of the present invention further comprises a supplement.

In another aspect, the present invention provides a method of treatment of hypoxia and related disorders, in which the method includes administering an effective amount of the formulation to the subject in need thereof. In a preferred embodiment, the aforesaid related disorders are related to oxidation and free radicals.

In another preferred embodiment, the use of the formulation in the treatment of hypoxia and related disorders is described. Yet in another preferred embodiment, the formulation is used for the manufacture of a medicament for the treatment of hypoxia and other related disorders. In another preferred embodiment, the aforesaid related disorders are related to oxidation and free radicals.

In another preferred embodiment, hypoxia includes blood hypoxia, cerebral hypoxia, tissue and cell hypoxia and other related disorders.

The invention according to another aspect provides a method of treating hypoxia and related disorders, wherein the method includes administering an effective amount of a plant extract formulation to the subject in need thereof. The formulation comprises a mixture of lemon oil, eucalyptus oil, basil oil, davana oil, rosewood oil, fennel oil, and citronella oil, with the corresponding relative ratio thereof being listed in Table 2.

In a preferred embodiment, the formulation further comprises a supplement. Yet in a more preferred embodiment, the supplement is corn oil and the corresponding relative ratio of the various components of the formulation in this embodiment is listed in Table 3.

The instant invention also provides a formulation for the treatment of hypoxia and other related disorders, and the relative ratio of the various components of the formulation is listed in Table 2 or 3. The instant invention also provides a use of the formulation for the manufacture of a medicament for the treatment of hypoxia and other related disorders, and the relative ratio of the various components of the formulation is listed in Table 2 or 3. In a preferred embodiment, the aforesaid related disorders are related to oxidation and free radicals.

In another preferred embodiment, hypoxia includes blood hypoxia, cerebral hypoxia, tissue and cell hypoxia and other related disorders.

In another aspect, the invention provides a method of enhancing human immunity in which the method includes administrating an effective amount of a plant extract formulation to a subject in need thereof and the formulation comprises citronella oil. In a preferred embodiment, the formulation further comprises lemon oil, eucalyptus oil, basil oil, davana oil, rosewood oil, fennel oil, or any combination thereof.

In yet another aspect of the instant invention, a method of treating skin related diseases is provided in which an effective amount of a plant extract formulation is administrated to a subject in need thereof. The formulation comprises citronella oil; in a preferred embodiment, the formulation further comprises lemon oil, eucalyptus oil, basil oil, davana oil, rosewood oil, fennel oil, or any combination thereof.

In another preferred embodiment, the skin disease includes inflammatory skin diseases, viral skin diseases, bacterial skin diseases, fungal skin diseases, and radiation-related skin diseases. In yet another more preferred embodiment, the skin disease includes rashes, acnes, and sunburn.

The invention in another aspect provides a method of enhancing skin quality wherein the method includes administrating an effective amount of a plant extract formulation to a subject in need thereof and the formulation comprises citronella oil. In a preferred embodiment, the formulation further comprises lemon oil, eucalyptus oil, basil oil, davana oil, rosewood oil, fennel oil, or any combination thereof.

In another preferred embodiment, the enhancement of skin quality includes restoration of skin flexibility, minimizing of skin pore size, whitening of skin, removal of dark-eye circle, and removal of wrinkles.

The present invention combines the lipid soluble molecules of different natural plant extracts to produce therapeutic effects on hypoxic disorders of blood cells. The time lag to onset of the drug action is short and results are good.

Additionally, the present invention can be applied topically because the natural small molecules can permeate across the skin and mucosa tissues (e.g. mucosa tissue of red blood cells, brain cells, skin cells, muscle cells, lung cells) for the modulation, restoration, and improvement of cells.

### DETAILED DESCRIPTION OF THE PREFERRED EMBODIMENTS

As used herein and in the claims, “comprising” means including the following elements but not excluding others. Further, “extract oil” as described herein is extracted or refined from the corresponding plant using routine or common method such as but not limited to distillation, pressing, dissolution, supercritical carbon dioxide extraction “Extract oil” per se refers to concentrated, lipophilic, and volatile oil comprising compounds that are derivatives of terpenes, ketones, and phenols. The term “blood hypoxia” includes but not limited to condition involving low circulating oxyhaemoglobin relative to deoxyhemoglobin, and conditions described by hypoxemic hypoxia and anemic hypoxia. The term “tissue and cell hypoxia” includes but not limited to hypoxia conditions in tissues or cells of organs such as liver, kidney, lung, heart, etc.

The term “relative ratio” of a particular component of the formulation refers to the ratio of the amount of that component to the amount of a selected base component in the formulation. For example, in Mixture 1 of the instant invention, fennel oil is selected as the base component and from Table 1, the relative ratio of lemon oil: fennel oil is 2-6:1. In other words, the amount of the lemon oil present in the formulation is 2-6 times more than that of the fennel oil. Thus, according to this mixing principle, the relative proportion by amount of each component in the formulation in the present invention is fixed, regardless of any changes in the total volume or the total weight of the formulation. In the same way, adding additional component(s) not specified in the formulation would not change the proportion of each component listed in the formulation relative to each other.

The present invention in one aspect provides a formulation with components and the corresponding relative ratio listed in Table 1, in which Mixtures 1, 2 and 3 describe three workable ranges of the relative ratio of the formulation. In the calculation of the relative ratio for the three Mixtures 1, 2, and 3 in this Table 1, fennel oil is selected as the base component.

TABLE 1

| | MIXTURE 1 | MIXTURE 2 | MIXTURE 3 |
|---|---|---|---|
| Components | Relative Ratio | Relative Ratio | Relative Ratio |
| Lemon oil | 2-6 | 2-5 | 2-4 |
| Eucalyptus oil | 5-9 | 5-8 | 5-7 |
| Basil oil | 3-6 | 3-5 | 3-4 |
| Davana oil | 1-4 | 1-3 | 1-2 |
| Rosewood oil | 1-4 | 1-3 | 1-2 |
| Fennel oil | 1 | 1 | 1 |
| Citronella oil | 6-12 | 6-11 | 6-9 |

In another preferred embodiment, the formulation as shown in Table 1 further comprises a supplement, and the supplement includes plant oils such as corn oil, soy bean oil, and olive oil.

In another aspect, the present invention provides a formulation as shown in Table 2 in which Mixtures 4, 5 and 6 describe three working ranges of the relative ratio of the formulation. In the calculation of the relative ratio for the three Mixtures 4, 5, and 6 in this Table 2, fennel oil is selected as the base component.

TABLE 2

| | MIXTURE 4 | MIXTURE 5 | MIXTURE 6 |
|---|---|---|---|
| Components | Relative Ratio | Relative Ratio | Relative Ratio |
| Lemon oil | 1-4 | 1-3 | 1-2 |
| Eucalyptus oil | 1-4 | 2-4 | 3-4 |
| Basil oil | 1-4 | 2-4 | 2-3 |
| Davana oil | Less than 3 | Less than 2 | Less than 1 |
| Rosewood oil | 1-4 | 1-3 | 1-2 |
| Fennel oil | 1 | 1 | 1 |
| Citronella oil | 2-5 | 2-4 | 3-4 |

In a preferred embodiment, the formulation as shown in Table 2 further comprises a supplement, and the supplement includes plant oils such as corn oil, soy bean oil, and olive oil.

In a further aspect of the instant invention, the present invention provides a formulation as shown in Table 3 in which Mixtures 7, 8 and 9 describe three working ranges of relative ratio of the formulation. In the calculation of the relative ratio for the three Mixtures 7, 8, and 9 in this Table 3, fennel oil is selected as the base component.

TABLE 3

| | MIXTURE 7 | MIXTURE 8 | MIXTURE 9 |
|---|---|---|---|
| Components | Relative ratio | Relative ratio | Relative ratio |
| Lemon oil | 1-4 | 1-3 | 1-2 |
| Eucalyptus oil | 1-4 | 2-4 | 3-4 |
| Basil oil | 1-4 | 2-4 | 2-3 |
| Davana oil | Less than 3 | Less than 2 | Less than 1 |
| Rosewood oil | 1-4 | 1-3 | 1-2 |
| Fennel oil | 1 | 1 | 1 |
| Citronella oil | 2-5 | 2-4 | 3-4 |
| Corn oil | 40-70 | 40-70 | 40-70 |

In another aspect, the present invention provides cleansing products such as shampoo, cream, soap, cosmetics or other skin care products such as lotion, ointment, cream and etc manufactured by using formulation as shown in Table 1, 2 or 3.

The invention is further defined by the following examples, which are not intended to limit the present invention.

### Example 1

### Study on Blood pH and Blood Hypoxia

In this study regarding oxygen carrying ability of red blood cells, the test item was the formulation as described in Mixture 3 listed in Table 1 (the formulation). First of all, healthy SD mice were fed with sodium nitrite (17 mg/kg) by gastric gavage for 7 consecutive days to induce the hypoxia animal model. The mice were then randomly distributed into 8 groups: (A) normal mice blank control group, (B) normal mice applied with the formulation group (82.5 μL/kg), (C) model mice applied with vitamin C (62.5 mg/kg) control group, (D) model mice applied with vitamin E (104 mg/kg) control group, (E) model mice applied with vegetable oil (82.5 μL/kg) control group, (F) model mice applied with high dose of the formulation (330 μL/kg), (G) model mice applied with medium dose of the formulation (165 μL/kg), (H) model mice applied with low dose of the formulation (82.5 μL/kg). There were 12 mice for each group.

On the 7^{th} day of hypoxia model induction, the formulation was applied topically on the mice in the formulation groups (B, F, G and H), and equal amount of vegetable oil was also topically applied on the mice in the group (E). Vitamins C and E were administrated by gastric gavage to mice in groups (C) and (D) respectively. 10 days after the application of the corresponding items, blood samples were taken in which blood pH, arterial oxygen partial pressure (pO₂), and blood oxygen were tested, and the results are as listed below:

TABLE 4
Value (Mean ± SD)

| | Types of | | Oxygen partial | |
|---|---|---|---|---|
| Group | mice | | pressure | Blood oxygen |
| (Dosage (mg/kg) | tested | Blood pH | (kPa) | (%) |
| (A) Blank Control | Normal | 7.406 ± 0.058 | 10.49 ± 0.82 | 93.6 ± 1.58 |
| (B) Formulation | Normal | 7.391 ± 0.057 | 10.91 ± 1.44 | 93.6 ± 2.5 |
| (82.5 μl/kg) | | | | |
| (C) Vitamin C Control | Hypoxia | 7.420 ± 0.036** | 10.51 ± 0.96** | 93.6 ± 1.3** |
| (62.5 mg/kg) | Model | | | |
| (D) Vitamin E Control | Hypoxia | 7.455 ± 0.049** | 10.76 ± 2.14** | 94.0 ± 2.1** |
| (104 mg/kg) | Model | | | |
| (E) Vegetable Oil | Hypoxia | 7.382 ± 0.059 | 7.33 ± 1.11 | 81.2 ± 8.3 |
| Control | Model | | | |
| (82.5 μl/kg) | | | | |
| (F) Formulation - | Hypoxia | 7.425 ± 0.049** | 9.60 ± 1.45** | 92.7 ± 2.0** |
| High Dosage | Model | | | |
| (330 μl/kg) | | | | |
| (G) Formulation - | Hypoxia | 7.418 ± 0.037* | 10.07 ± 1.23** | 92.6 ± 2.5** |
| Medium Dosage | Model | | | |
| (165 μl/kg) | | | | |
| (H) Formulation - | Hypoxia | 7.417 ± 0.061* | 10.37 ± 0.92** | 93.1 ± 2.3** |
| Low Dosage | Model | | | |
| (82.5 μl/kg) | | | | |

Note:
As compared with plant oil control group,
*p < 0.05,
**p < 0.01

The above results showed that the formulation of the present invention can affect blood pH. The blood pH of model mice in group (E) is lower than that in normal mice group (A). Further, the blood pH of the model mice in all the three formulation groups (F, G and H) have been improved after the application of the formulation. Thus, the results suggested that the formulation can significantly normalize the blood pH level.

Under hypoxia condition, the blood pH is varied because of the accumulation of lactic acid, which in turn leads to a decrease of oxygen binding ability in red blood cell due to its functional changes. Thus, the above test results demonstrated that the formulation of the present invention can modulate blood pH under blood hypoxia condition, and restore functions of red blood cells due to the changes in blood pH. In addition, the oxygen binding ability of hemoglobin can be improved, restoring malfunctioned red blood cells and eliminating diseases due to cell apoptosis and abnormal oxygen metabolism.

Further, the aforesaid results show that the formulation of the present invention, through skin application, can increase oxygen partial pressure and blood oxygen of hypoxic mice. In short, the results indicate that the present invention is useful in treating blood hypoxia or the related disorders thereof. Similar effects can be achieved using Mixtures 1 and 2 (as listed in Table 1).

### Example 2

### Study on Anti-Oxidation and Anti-Free Radicals Treatment for Hemoglobinopathy

Sodium nitrite is a strong oxidizing agent which can be found in a great variety of food. Upon entering into body in large quantity, sodium nitrite would lead to methemoglobinemia (MetHb) in which hemoglobins cannot carry and release oxygen normally, leading to tissue/cells hypoxia and other poisoning symptoms.

In this study regarding oxygen carrying ability of red blood cells, the test item was the formulation as described in Mixture 3 listed in Table 1 (the formulation). The positive control drug was Nimodipine tablets manufactured by Guangdong Huanan Pharmacy Limited. The negative control item was vegetable oil readily available from markets.

This test was designed according to the relevant provisions of The Management of Medicine Registration issued by the State Food and Drug Administration (SFDA) of the People's Republic of China, and The Assemble of Guiding Principle of Pre-clinical Research on New Drugs (in Western Medicine) from the Bureau of Drug Administration, Ministry of Public Health, People's Republic of China. Five test groups were designed for this test, namely the negative control group (A), the positive control medicine group (B), and three groups administrated with the test item at the high (C), medium (D), and low (E) dosage levels. SPF class NIH mice were randomly distributed into 10-12 pieces (male quasi) in each group. The design of experiment was described in Table 5.

TABLE 5

| Group | Treatment | Dosage | | Route |
|---|---|---|---|---|
| A | Negative | 0.96 | mL/kg | Skin application |
| | Control | | | |
| B | Positive | 62 | mg/kg | Gastric gavage |
| | Control | | | |
| C | Formulation | 0.96 | mL/kg | Skin application |
| D | Formulation | 0.48 | mL/kg | Skin application |
| E | Formulation | 0.24 | mL/kg | Skin application |

In establishing the methemoglobinemia model, a 2% sodium nitrite was injected into abdomen of the mice (male mice: 240 mg/kg, 12 mL/kg; female mice: 260 mg/kg, 13 mL/kg). Stopwatch was immediately started to record time and simultaneously, vegetable oil or the test item was applied to the mice of the respective group. Mice in the positive control group I (group B-I) were administered with Nimodipine through gastric gavage an hour before model was established, while those in the positive control group II were administered Nimodipine through gastric gavage immediately upon the setting up of the model. All the groups were observed for 90 minutes and the survival duration of each animal was recorded. The results are shown as follows:

TABLE 6
Value (Mean ± SD)

| | | Survival Duration | Prolonged |
|---|---|---|---|
| Group | Gender | (min) | Percentage (%) |
| A | Male | 29.84 ± 7.68 | — |
| | Female | 23.32 ± 7.40 | — |
| | Male quasi | 31.00 ± 7.16 | — |
| B-I | Male quasi | 48.44 ± 21.36* | 56.26 |
| B-II | Male quasi | 39.10 ± 17.30 | 26.24 |
| C | Male | 58.64 ± 28.55** | 96.52 |
| | Female | 37.47 ± 20.93 | 60.69 |
| D | Male | 37.55 ± 21.11 | 25.86 |
| | Female | 28.46 ± 12.35 | 22.03 |
| E | Male | 30.70 ± 12.15 | 2.87 |
| | Female | 25.63 ± 8.06 | 9.91 |

Note:
As compared with negative control group of the corresponding gender,
*P < 0.05,
**P < 0.01

As shown in the Table 6, the results indicate the survival duration of the mice is significantly extended, and that of the male mice of the high dosage group (group C) is noticeably extended by 96%. Thus, the formulation of the present invention can significantly resist against and treat for sodium nitrite induced blood disorder in mice, prolonging their survival time. The formulation of the present invention is thus shown to be effective in treating blood hypoxia and tissue/cells hypoxia through its anti-oxidative and anti-free radical properties. Similar effects can be achieved using Mixtures 1 and 2 (as listed in Table 1).

### Example 3

### Study on Cerebral Ischemia

In this study regarding oxygen carrying ability of red blood cells, the test item was the formulation as described in Mixture 3 listed in Table 1 (the formulation). The positive medicine control was Buchang Naoxintong capsules manufactured by Xianyang Buchang Pharmacy Co. Ltd. The negative control item was vegetable oil readily available from markets.

This test was designed according to the relevant provisions of The Management of Medicine Registration issued by the State Food and Drug Administration (SFDA) of the People's Republic of China, and The Assemble of Guiding Principle of Pre-clinical Research on New Drugs (in Western Medicine) from the Bureau of Drug Administration, Ministry of Public Health, People's Republic of China. Six test groups were designed for this test, namely mice model control group (A), sham operation group (B), positive medicine control group (C) and three groups administered with the test item at the high (D), medium (E), and low (F) dosage. SPF class SD mice were randomly distributed, male quasi. Among them, for the study on the influence on their behavior, there were 16-20 mice per group; for the study on the influence on water content of brain tissue, there were 8-10 mice per group; for the study on the influence on infarction volume in brain tissue, there were 8-10 mice per group. The design of experiment was described in Table 7.

TABLE 7

| Group | Treatment | Dosage | | Route |
|---|---|---|---|---|
| A | Negative | 0.4 | mL/kg | Skin application |
| | Control | | | |
| B | Negative | 0.4 | mg/kg | Skin application |
| | Control | | | |
| C | Positive | 480 | mg/kg | Gastric gavage |
| | Control | | | |
| D | Formulation | 0.4 | mL/kg | Skin application |
| E | Formulation | 0.2 | mL/kg | Skin application |
| F | Formulation | 0.1 | mL/kg | Skin application |

Mice in model control group (A) and sham operation group (B) were topically applied with vegetable oil, whereas those in the positive medicine group (C) received Buchang Naoxintong through gastric gavage. Mice in groups (D), (E), and (F) received the test item by topical application once a day for seven consecutive days. Focal cerebral ischemia model was set up by adopting a thread embolism method 1 hour after the final administration of the above mentioned items. A 10% chloral hydrate solution was abdominally injected into the anesthetized rats (3 mL/kg.bw). First, an opening in the centre of the neck was made, and the left side neck master artery near the heart, as well as neck exterior artery and branch artery, were ligated and separated. Then, the left side neck interior artery, passing through this artery down to the wing artery, was separated and ligated at the root of that branch. A thread was set near the neck exterior artery. An artery clamp was placed at the far end of the neck interior artery and master artery. A cut was made near the neck exterior artery at the crotch of the master artery. Then a 4.0 nylon thread was inserted to a depth of 18.5±1.5 mm The thread embolism was extended from the neck interior artery through the skull to the cerebrum to block all the blood supply from the cerebrum artery. The artery clamp was removed, the thread was tightened up with 1 cm of its left outside, and the cut was stitched up and waited for 2 hour. The affected area was reperfused 2 hours after the focal cerebral ischemia was set by slightly pulling the thread until it met with resistance which signified that it had come to the opening of neck exterior artery and blood would begin to re-circulate. For the sham operation group, all steps were the same except for the step of inserting the thread.

The survived mice were reperfused for 24 hours, their behavior was monitored and the following three tests were carried out:

(I) Test on the Influence on Behavior—based on a 5-point standard Zea Longa reference: 0 point—mice were normal with no neural injury; 1 point—mice were unable to stretch out their front claws; 2 points—mice would circle around the outer perimeter; 3 points—mice would lean towards the opposite sides; 4 points—mice were unable to walk on its own with loss of consciousness.

(II) Test on the Influence on Water Content of Brain Tissue—half of the mice from each group (8-10 pieces) were retrieved to dissect, the brain was removed and divided into halves, and the left and right hemispheres were wet-weighed. Then the two hemispheres were placed in a 120° C. oven for 48 hours and dry-weighed upon achieving a constant weight. The water content of the brain tissue was calculated according to the following formula: Water Content in the brain tissue (%)=(wet weight−dry weight)/wet weight×100%.

(III) Test on the Influence on Infarction Volume in Brain Tissue—the heads of the other half of the mice (8-10 pieces) were quickly severed to retrieve the brain (with olfactory bulb, cerebellum and low-positioned brain stem being removed). The coronary brain was sliced into 5 or 6 pieces at about 2 mm in thickness and promptly placed into a 2% TCC solution with incubation at 37° C. for 40 minutes. The infarct region would become white while the non-infarct region would become red. Pictures were taken and recorded with a digital camera. Medbrain 2.0 software (Nanjing Medease Science and Technology Co., Ltd.) was used to determine the total volume of the brain slices and the infarct regions and to calculate the percentage of infarct region among the entire brain tissue. The results are as follows:

TABLE 8
Value (Mean ± SD)

| | | | | III. Influence on Brain |
|---|---|---|---|---|
| | | II. Influence on Water Content | | Infarct Volume of |
| | I. Influence on | of Brain Tissue | | Cerebral Ischemia |
| | Behavior | Left Brain | Right Brain | Percentage of Infarction |
| | Behavior | Hemisphere | Hemisphere | Volume in the Overall |
| Group | Rating | (%) | (%) | Brain Tissues (%) |
| A | 2.71 ± 0.64* | 79.36 ± 0.22 | 78.87 ± 0.74 | 36.74 ± 7.81* |
| B | 0 | 78.53 ± 0.14 | 78.39 ± 0.33 | 3.18 ± 0.90 |
| C | 2.26 ± 0.75** | 79.26 ± 0.65 | 78.53 ± 0.27 | 22.20 ± 3.65** |
| D | 2.08 ± 0.65** | 78.82 ± 0.41 | 78.44 ± 0.19 | 25.35 ± 5.16** |
| E | 2.16 ± 0.53** | 78.83 ± 0.56 | 78.52 ± 0.39 | 19.43 ± 1.42** |
| F | 1.97 ± 0.70** | 79.56 ± 0.21 | 78.51 ± 0.38 | 18.96 ± 6.56** |

Note:
As compared with negative control group,
*P < 0.01;
as compared with sham group,
**P < 0.01

As shown in Table 8, the results show that the formulation of the present invention can improve the rating of the neural behavior of the tested mice and significantly reduce the neural damages caused by focal cerebral ischemia and reperfusion injuries in mice. Further, the formulation of the present invention in medium and high dosage groups (D and E) exhibit a tendency to reduce the water content of the brain tissue on the ischemia hemisphere. This result indicates that the instant invention can reduce the injuries caused by cerebral ischemia and reperfusion, and alleviate hydrocephalus in mice. When compared with mice model control group, the volume of infarct region in the group receiving the formulation of the present invention was reduced by at least 30%, showing that the present invention can significantly reduce cerebral infarction caused by focal cerebral ischemia and reperfusion injuries, reduce the damage of brain cells, and protect the neural cells.

In summary, the formulation of the present invention can alleviate the neurological deficits (behavioral aberrance) caused by focal cerebral ischemia and reperfusion injuries. It can also reduce brain edema on the ischemic hemisphere and scale down the volume of cerebral infarction. Therefore, the results indicate that the present invention can treat and protect the brain tissue suffering from focal cerebral ischemia and reperfusion injuries. Similar effects can be achieved using Mixtures 1 and 2 (as listed in Table 1).

### Example 4

### Study on Global Cerebral Ischemia

In this study regarding oxygen carrying ability of red blood cells, the test item was the formulation as described in Mixture 3 listed in Table 1 (the formulation). The positive medicine control was Nimodipine injection liquid manufactured by Tianjin Jinyao Amino Acid Co., Ltd. The negative control item was vegetable oil readily available from markets.

Five test groups were designed for this test, namely the negative control group (A), the positive control medicine group (B), and three groups administrated with the test item at the high (C), medium (D), and low (E) dosage levels. SPF class NIH mice were randomly distributed into 14 pieces in each group. The design of experiment was described in Table 9.

TABLE 9

| Group | Treatment | Dosage | Route |
|---|---|---|---|
| A | Negative | 1.0 mL/kg | Skin application |
| | Control | | |
| B | Positive | 2.0 mg/kg | Hypodermic |
| | Control | | injection |
| C | Formulation | 1.0 mL/kg | Skin application |
| D | Formulation | 0.5 mL/kg | Skin application |
| E | Formulation | 0.25 mL/kg | Skin application |

1. Preliminary Test

Mice in negative control group (A) were topically applied with vegetable oil, whereas those in positive control group (B) were administrated with Nimodipine by hypodermic injection. Mice in groups (C), (D), and (E) received the test item through skin administration. All the five groups received the corresponding item once a day for two consecutive days. 60 minutes after the final administration of the test item and vegetable oil to the mice of the corresponding groups, and 30 minutes after the final administration of Nimodipine to the mice of the positive medicine control group, the heads of mice were rapidly severed from the back of ear root with a pair of sharp scissors (decapitation method). The gasping duration and gasping frequency were immediately recorded with a stopwatch. Similar effects can be achieved using Mixtures 1 and 2 (as listed in Table 1).

The result of the preliminary tests showed that, when comparing with the negative control group, the three formulation groups show different levels of increment of the gasping frequency after decapitation, although none of the formulation groups show extension in the gasping duration.

2. Formal Test

Mice in negative control group (A) were topically applied with vegetable oil, whereas those in positive control group (B) were administrated with Nimodipine by hypodermic injection once a day for 2 consecutive days. Mice in groups (C), (D), and (E) received the test item through skin administration once a day for 7 consecutive days.

60 minutes after the final administration of the test item and vegetable oil to the mice of the corresponding groups, and 30 minutes after the final administration of Nimodipine to the mice of the positive medicine control group, the heads of mice were rapidly severed from the back of ear root with a pair of sharp scissors (decapitation method). The gasping duration and gasping frequency were immediately recorded with a stopwatch The results are shown as follows:

TABLE 10
Value (Mean ± SD)

| | Gasping Duration | | | |
|---|---|---|---|---|
| | | Extension | Gasping Frequency | |
| Group | Time (sec) | Rate (%) | Times | Increment Rate (%) |
| A | 19.1 ± 2.6 | — | 11.6 ± 2.2 | — |
| B | 24.6 ± 4.6** | 28.6 | 14.5 ± 3.6* | 25.6 |
| C | 18.7 ± 2.0 | — | 13.6 ± 1.8* | 17.3 |
| D | 17.9 ± 1.7 | — | 14.4 ± 1.5** | 24.3 |
| E | 18.9 ± 2.8 | — | 14.5 ± 1.6** | 25.0 |

Note:
As compared with negative control group,
*P < 0.05,
**P < 0.01

The decapitation process would stop the mice cerebral blood supply, but the blood and nutritional substances that retained in their brains could still allow them to gasp regularly for a short while. Taking this as the evaluation index and the protective ability of the formulation of the present invention against cerebral ischemia can be observed. Any medicine that could reduce cerebral oxygen consumption would extend the mice gasping duration. In the results above, the formulation of the present invention demonstrated a noticeable increase in gasping frequency after decapitation, although it does not show any extension in the gasping duration. This indicates that the present invention can protect the mice suffering from acute cerebral ischemic hypoxia by increasing the breathing frequency (an increase in the oxygen supply) and the density of blood oxygen, instead of reducing oxygen and energy consumption of the cerebral tissue. Therefore, the results show the instant invention has pharmacological protective effects on the tested mice that suffered from acute cerebral ischemic hypoxia. Similar effects can be achieved using Mixtures 1 and 2 (as listed in Table 1).

### Example 5

### Study on Memory Consolidated Handicap

Sodium nitrate, a cerebral hypoxic agent, was used to establish this memory consolidated handicap model. Due to its strong oxidation property, it could convert low value iron in the hemoglobin into high value iron and thus affects the hemoglobin to lose its anti-oxidative ability, resulting in Methemoglobinemia disease. Brain tissue is the most sensitive tissue to hypoxia and it would be injured once it experienced memory consolidated handicap. Therefore, in the establishment of this model, the applying of sodium nitrate would affect the space recognition ability of the mice.

In this study regarding oxygen carrying ability of red blood cells, the test item was the formulation as described in Mixture 3 listed in Table 1 (the formulation). The positive control medicine was vitamin C injection liquid manufactured by Guangzhou Tianxin Pharmaceutical Company Limited. The negative control item was vegetable oil readily available from markets.

This test was designed according to the relevant provisions of The Management of Medicine Registration issued by the State Food and Drug Administration (SFDA) of the People's Republic of China, and The Assemble of Guiding Principle of Pre-clinical Research on New Drugs (in Western Medicine) from the Bureau of Drug Administration, Ministry of Public Health, People's Republic of China. Five test groups were designed for this test, namely the negative control group (A), the positive control medicine group (B), and three groups administrated with the test item at the high (C), medium (D), and low (E) dosage levels. SPF class NIH mice were randomly distributed into 10 pieces (male quasi) in each group. The design of experiment was described in Table 11.

TABLE 11

| Group | Treatment | Dosage | Route |
|---|---|---|---|
| A | Negative | 0.48 mL/kg | Skin application |
| | Control | | |
| B | Positive | 1.04 mg/kg | Intramuscular |
| | Control | | injection |
| C | Formulation | 0.48 mL/kg | Skin application |
| D | Formulation | 0.24 mL/kg | Skin application |
| E | Formulation | 0.12 mL/kg | Skin application |

Each group of mice was continuously trained for 5 days, once in the morning and once in the afternoon, for 60 seconds in each training session. Before the training session on the afternoon of the 5^{th} day, sodium nitrite was administered at dose level of 160 mg/kg to the mice through hypodermic injection to induce the mice to experience memory consolidated handicap symptom. 30 minutes after medication, the water maze test was carried out. The aforesaid test was then repeated on the following morning.

The results are as follows:

TABLE 12
Value (Mean ± SD)

| | Time to Reach the Platform (sec) | | |
|---|---|---|---|
| Group | Day 5, Morning | Day 5, Afternoon | Day 6, Morning |
| A | 35.6 ± 24.4 | 48.6 ± 17.6 | 60.0 ± 0.0 |
| B | 42.0 ± 24.3 | 46.9 ± 15.5 | 41.7 ± 21.1* |
| C | 41.8 ± 17.6 | 37.7 ± 21.1 | 40.3 ± 22.2* |
| D | 44.3 ± 18.1 | 40.3 ± 23.0 | 45.2 ± 23.8 |
| E | 35.2 ± 23.7 | 37.7 ± 26.0 | 37.3 ± 20.9* |

Note:
As compared with negative control group,
*P < 0.05.

The results above show that, as compared to the results from the negative control group, the formulation according to the instant invention could shorten the time for the mice to reach the platform and thus improve the space recognition learning ability of the mice. Therefore, the results indicate that the instant invention could reduce the brain injury of the mice caused by sodium nitrite induced methemoglobinemia and simultaneously, the instant invention could adopt its anti-oxidative properties to reduce the symptoms of cerebral hypoxia. Similar effects can be achieved using Mixtures 1 and 2 (as listed in Table 1).

### Example 6

### Study on Hypobaric Hypoxia

In this study regarding oxygen carrying ability of red blood cells, the test item was the formulation as described in Mixture 3 listed in Table 1 (the formulation). The positive control medicine was Propranolol manufactured by Tianjin Lisheng Pharmacy Co., Ltd. The negative control item was vegetable oil readily available from markets.

Five test groups were designed for this test, namely the negative control group (A), the positive control medicine group (B), and three groups administrated with the test item at the high (C), medium (D), and low (E) dosage levels. SPF class NIH mice were randomly distributed into 12 pieces in each group. The design of experiment was described in Table 13.

TABLE 13

| Group | Treatment | Dosage | Route |
|---|---|---|---|
| A | Negative | 0.96 mL/kg | Skin application |
| | Control | | |
| B | Positive | 26 mg/kg | Gastric gavage |
| | Control | | |
| C | Formulation | 0.96 mL/kg | Skin application |
| D | Formulation | 0.48 mL/kg | Skin application |
| E | Formulation | 0.24 mL/kg | Skin application |

One hour after administrating the positive control medicine, and 30 minutes after applying the test item or the negative control in the corresponding groups, the mice (6 mice/batch, 2 batches/group) were put in consecutive order into the vacuum drier connected with a suction (built in soda lime to absorb CO₂ and water). The suction process was started until the air pressure dropped to a certain negative pressure (Male mouse: 580 mmHg; female mouse: 600 mmHg). Then, stopwatch was used to record the survival duration and the number of survivals in each group within 60 minutes. The results are as follows:

TABLE 14
Value (Mean ± SD)

| | | Survival | Prolonged | Survival |
|---|---|---|---|---|
| | | Duration | Percentage | Percentage |
| Group | Gender | (min) | (%) | (%) |
| A | Male | 31.33 ± 14.35 | — | 16.7 |
| | Female | 24.91 ± 17.63 | — | 8.3 |
| | Male quasi | 24.19 ± 14.52 | — | 8.3 |
| B | Male quasi | 50.40 ± 16.33** | 108.4 | 41.7 |
| C | Male | 55.14 ± 13.07** | 76.03 | 66.7* |
| | Female | 30.60 ± 24.81 | 22.82 | 25.0 |
| D | Male | 52.08 ± 15.44** | 66.25 | 75.0 |
| | Female | 48.74 ± 20.35** | 95.64 | 66.7 |
| E | Male | 37.09 ± 23.20 | 18.41 | 33.3 |
| | Female | 30.70 ± 19.43 | 23.23 | 16.7 |

Note:
As compared with negative control group of the corresponding gender,
*P < 0.05,
**P < 0.01

Putting the mice into a sealed container with the subsequent removal of part of the air from the container would create a low-pressure hypoxic environment in which the mice would die. The results above show that the formulation according to the instant invention could noticeably enhance the mice's tolerance ability against the falling of partial pressure and oxygen reduction, and thus increase their survival duration for the mice and reduce their mortality rate. The results also indicate that this invention takes its effect by enhancing the ability of the animal's oxygen utilization and increasing the oxygen supply to cells, which could in turn increase the animals' oxygen partial pressure in blood as well as their anti-oxidization response ability. Similar effects can be achieved using Mixtures 1 and 2 (as listed in Table 1).

### Example 7

### Study on Cerebral Circulatory Disturbance Hypoxia

In this study regarding oxygen carrying ability of red blood cells, the test item was the formulation as described in Mixture 3 listed in Table 1 (the formulation). The positive control medicine was Nimodipine injection liquid manufactured by Tianjin Jinyao Amino Acid Co., Ltd. The negative control item was vegetable oil readily available from markets.

Five test groups were designed for this test, namely the negative control group (A), the positive control medicine group (B), and three groups administrated with the test item at the high (C), medium (D), and low (E) dosage levels. SPF class NIH mice were randomly distributed into 12 to 14 pieces in each group. The design of experiment was described in Table 15.

TABLE 15

| Group | Treatment | Dosage | Route |
|---|---|---|---|
| A | Negative | 1.0 mL/kg | Skin application |
| | Control | | |
| B | Positive | 2.0 mg/kg | Hypodermic |
| | Control | | injection |
| C | Formulation | 1.0 mL/kg | Skin application |
| D | Formulation | 0.5 mL/kg | Skin application |
| E | Formulation | 0.25 mL/kg | Skin application |

Mice in negative control group (A) were topically applied with vegetable oil, whereas those in positive control group (B) were administrated with Nimodipine by hypodermic injection. Mice in groups (C), (D), and (E) received the test item through skin administration. All the five groups received the corresponding item once a day for 5 consecutive days. 50 minutes after the final administration of the test item, and 30 minutes after the final administration of Nimodipine, the mice were given an intraperitoneal injection of Urethane (8%, 0.8 g/kg.bw, 10 mL/kg.bw) as anesthetics. The mice then underwent a separation of the left and right common carotid artery as well as the vagus, and were separately ligated with a No. 4 suture. Survival duration for the mice were immediately recorded with a stopwatch, with results as follows:

TABLE 16

| | | Prolonged |
|---|---|---|
| Group | Survival Duration (min) | Percentage (%) |
| A | 4.2 ± 1.6 | — |
| B | 55.4 ± 15.2** | 1224.3 |
| C | 6.7 ± 3.9* | 59.1 |
| D | 7.3 ± 4.2* | 75.5 |
| E | 4.8 ± 2.4 | 13.3 |

Note:
As compared with negative control group,
*P < 0.05,
**P < 0.01

The brain is the organ that reserves the least energy and oxygen but consumes the most of it. However, oxygen supply mostly depends on the blood's oxygen content and volume of blood flowing to the tissue vessels. If the body experiences global or local circulatory disruptions, it will slow down the speed of blood flow, which results in a drop or a stop of blood flowing and leads to stagnant hypoxia. The results above show that the formulation according to the instant invention can noticeably enhance the mice's tolerance ability against cerebral ischemic hypoxia, and thus increase the survival duration for the mice suffering from cerebral ischemic hypoxia. This also indicates that this invention acts by increasing the oxygen content in the blood and/or blood flow to the tissue vessels. Thus, the instant invention can enhance the mice's tolerance ability and protective capabilities against cerebral ischemic hypoxia within a fixed dosage range. So, treatment of cerebral ischemic hypoxia can be observed for the formulation in accordance with the instant invention. Similar effects can be achieved using Mixtures 1 and 2 (as listed in Table 1).

### Example 8

### Study on Athletic Performance

There were 7 participants (5 male, 2 female) in this study and they were applied with the formulation according to Mixture 3 listed in Table 1 (the formulation). 0.6 mL of the formulation was applied on their chests and 60 minutes later, all of the participants carried out tests on discus, javelin, weight throw, and shot put. Their results were recorded and compared with their respective best ever records. 6 of the 7 participants carried injuries with them before the administration of the formulation according to the present invention.

The results of this study were shown in Table 17. In short, upon application of the formulation, participants were able to maintain their best ever records or even make breakthroughs over their own records. Therefore, enhancement and improvement on human physical strength and athletic performance can be observed for the formulation of the instant invention. Similar effects can be achieved using Mixtures 1 and 2 (as listed in Table 1).

TABLE 17

| | |
|---|---|
| 1. | 57% Participants made breakthroughs over their best records |
| 2. | 43% Participants maintained their best records |
| 3. | 100% Participants experienced enhancement in their physical strength |

### Example 9

### Study on Athletic Performance

There were 16 participants (11 male, 5 female) in this study and they were applied with the formulation according to Mixture 3 listed in Table 1 (the formulation) for every 12 hours for a total of three times. 0.6 mL of the formulation was applied on their chests in each administration and 60 minutes after the final administration, all of the participants carried out tests on weightlifting. Their results were recorded and compared with their respective best ever records. All of the 16 participants carried injuries with them before the administration of the formulation according to the present invention.

The results of this study were shown in Table 18. In short, upon application of the formulation, participants were able to maintain their best ever records or even make breakthroughs over their own records. Therefore, enhancement and improvement on human physical strength and athletic performance can be observed for the formulation of the instant invention. Similar effects can be achieved using Mixtures 1 and 2 (as listed in Table 1).

TABLE 18

| | |
|---|---|
| 1. | 43% Participants made breakthroughs over their best records |
| 2. | 44% Participants maintained their best records |
| 3. | 100% Participants experienced enhancement in their physical strength |

### Example 10

### Study on High Altitude Hypoxia

This test was carried out in highlands with altitudes of 3100-3600 meters. The ten participants (4 male, 6 female) had demonstrated different degrees in symptoms such as rapid breathing, chest tightness, dizziness, nausea, and rapid pulse. Within the 10-60 minutes upon the onset of the aforesaid symptoms, the formulation according to Mixture 6 listed in Table 2 (the formulation) was applied to the participants as follows: 20 droplets (about 0.6 mL) and 5 droplets (about 0.15 mL) of the formulation were respectively applied on chest and the scalp of each participant. The results of the test were recorded on Table 19.

TABLE 19

| | | | Blood Oxygen | | Respiration Frequency | | Heartbeat | |
|---|---|---|---|---|---|---|---|---|
| | | | Saturation (%) | | (Per minute) | | (Per minute) | |
| | | | Prior to | 15 minutes | Prior to | 15 minutes | Prior to | 15 minutes |
| No | Gender | Age | Usage | after Usage | Usage | after Usage | Usage | after Usage |
| 1 | M | 28 | 93 | 99 | 28 | 19 | 86 | 67 |
| 2 | M | 26 | 90 | 98-99 | 28 | 20 | 81 | 69 |
| 3 | M | 32 | 87 | 99 | 31 | 23 | 91 | 75 |
| 4 | M | 34 | 89 | 99 | 33 | 19 | 87 | 69 |
| 5 | F | 38 | 85 | 97-99 | 34 | 25 | 90 | 74 |
| 6 | F | 37 | 89 | 99-100 | 32 | 23 | 81 | 72 |
| 7 | F | 35 | 84 | 99-100 | 29 | 23 | 83 | 75 |
| 8 | F | 34 | 85 | 98 | 30 | 19 | 87 | 71 |
| 9 | F | 27 | 85 | 99 | 31 | 21 | 83 | 64 |
| 10 | F | 28 | 87 | 98-99 | 31 | 25 | 84 | 67 |

In short, as shown in Table 20, improvement in blood oxygen saturation, respiration frequency and heartbeat were all demonstrated among all the participants. Similar effects can be achieved using Mixtures 4 and 5 (as listed in Table 2).

TABLE 20

| | |
|---|---|
| 1. | 100% Improvement on chest tightness, dizziness, nausea (10/10) |
| 2. | 100% Improvement on blood oxygen saturation (10/10) |
| 3. | 100% Improvement on heartbeat (10/10) |
| 4. | 100% Improvement on respiration frequency (10/10) |

### Example 11

### Study on High Altitude Hypoxia

This test was carried out in highlands with altitudes of 3000-3500 meters. The eight participants (5 male, 3 female) had demonstrated different degrees in symptoms such as rapid breathing, chest tightness, dizziness, nausea, and rapid pulse. Within the 10-60 minutes upon the onset of the aforesaid symptoms, the formulation according to Mixture 9 listed in Table 3 (the formulation) was applied to the participants as follows: 15 droplets (about 0.45 mL) and 5 droplets (about 0.15 mL) of the formulation were respectively applied on chest and the scalp of each participant. The results of the test were recorded on Table 21.

TABLE 21

| | | | Blood Oxygen | | Respiration Frequency | | Heartbeat | |
|---|---|---|---|---|---|---|---|---|
| | | | Saturation (%) | | (Per minute) | | (Per minute) | |
| | | | Prior to | 15 minutes | Prior to | 15 minutes | Prior to | 15 minutes |
| No | Gender | Age | Usage | after Usage | Usage | after Usage | Usage | after Usage |
| 1 | M | 32 | 92 | 99 | 27 | 18 | 89 | 69 |
| 2 | M | 37 | 89 | 98-99 | 28 | 20 | 81 | 72 |
| 3 | M | 43 | 89 | 99 | 30 | 21 | 90 | 75 |
| 4 | M | 26 | 93 | 99 | 25 | 17 | 79 | 69 |
| 5 | M | 47 | 87 | 97-99 | 32 | 20 | 91 | 73 |
| 6 | F | 36 | 91 | 99-100 | 30 | 19 | 78 | 69 |
| 7 | F | 32 | 89 | 99-100 | 27 | 19 | 83 | 71 |
| 8 | F | 38 | 87 | 98 | 30 | 18 | 87 | 71 |

In short, as shown in Table 22, improvement in blood oxygen saturation, respiration frequency and heartbeat were all demonstrated in all the participants. Similar effects can be achieved using Mixtures 7 and 8 (as listed in Table 3).

TABLE 22

| | |
|---|---|
| 1. | 87.5% Improvement on chest tightness (7/8) |
| 2. | 87.5% Improvement on dizziness (7/8) |
| 3. | 75% Improvement on nausea (6/8) |
| 4. | 100% Improvement on blood oxygen saturation (8/8) |
| 5. | 100% Improvement on heartbeat (8/8) |
| 6. | 100% Improvement on respiration frequency (8/8) |

### Example 12

### Study on Immunity

There were 24 participants (12 male, 12 female) with an average age of 9-11 in this study. They were applied with the formulation according to Mixture 1 listed in Table 1 or Mixture 4 listed in Table 2 (the formulation) twice daily for a total of 30 consecutive days. 0.6 mL of the formulation was applied on their chests in each administration. The results were recorded in Table 23.

TABLE 23

| | |
|---|---|
| 1. | 100% Male participants experienced less cold and/or flu symptoms |
| 2. | 100% Female participants experienced less cold and/or flu symptoms |

As observed from Table 23 above, upon application of the formulation, participants experienced less cold and/or flu symptoms. Therefore, the formulation of the instant invention is proved to be useful for the enhancement on human immunity. Similar effects can be achieved using Mixtures 2 and 3 as listed in Table 1 or Mixtures 5 and 6 as listed in Table 2.

### Example 13

### Study on Treatment of Skin Disease

In this study, there were 46 participants (20 male, 26 female) in which 8 of them had acnes problem, 13 of them had rashes problems, and 25 of them had sunburn problems. They were applied with the formulation according to Mixture 1 listed in Table 1 or Mixture 4 listed in Table 2 (the formulation) twice daily, once in the morning and evening respectively. 0.6 mL of the formulation was applied on the affected part(s) of the body in each administration. The results were recorded in Table 24.

TABLE 24

| | |
|---|---|
| 1. | 90% participants with acnes experienced improvement in their |
| | acne problems within 20 minutes upon the first administration |
| 2. | 90% participants with rashes experienced improvement in their |
| | rash problems within 30 minutes upon the first administration |
| 3. | 90% participants with sunburn experienced improvement in their |
| | sunburn problems within 10 minutes upon the first administration |

In short, upon application of the formulation, participants experienced improvement in their skin problems. Therefore, the formulation of the instant invention is proved to be useful in the treatment of skin diseases in human. Similar effects can be achieved using Mixtures 2 and 3 as listed in Table 1 or Mixtures 5 and 6 as listed in Table 2.

### Example 14

### Study on Skin Quality Enhancement

There were 600 participants (270 male, 330 female) in this study and they were applied with the formulation according to Mixture 1 listed in Table 1 or Mixture 4 listed in Table 2 (the formulation) twice daily, once in the morning and evening respectively. 0.6 mL of the formulation was applied on the needed part(s) of the body in each administration. The results were recorded in Table 25.

TABLE 25

| | |
|---|---|
| 1. | 95% participants experienced restoration of skin flexibility |
| 2. | 95% participants experienced minimizing of skin pore size |
| 3. | 95% participants experienced whitening of skin |
| 4. | 65% participants experienced improvement in their dark-eye circle |
| | problems and/or removal of their dark-eye circle problems |
| 5. | 87% participants experienced removal of their wrinkles |

In short, upon application of the formulation, participants experienced enhancement in their skin quality. Therefore, the formulation of the instant invention is proved to be useful for the enhancement in skin quality, such as restoration of skin flexibility, minimizing of skin pore size, whitening of skin, removal of dark-eye circle, and removal of wrinkles. Similar effects can be achieved using Mixtures 2 and 3 as listed in Table 1 or Mixtures 5 and 6 as listed in Table 2.

The preferred embodiments of the present invention are thus fully described. Although the description referred to particular embodiments, it will be clear to one skilled in the art that the present invention may be practiced with variation of these specific details. Hence this invention should not be construed as limited to the embodiments set forth herein.

For example, the formulation of the present invention can be applied topically, applied by inhaler, or taken orally upon dilution of the formulation. The formulation of the present invention can also be manufactured as different form such as spray, patches, cream, ointment, powder, capsule, or liquid. Further, the supplement of the formulation of the present invention includes the aforesaid oils but not excludes any other lipophilic substances as supplement.

What is claimed is:

1. A plant extract formulation comprising a mixture of
a) lemon oil;
b) eucalyptus oil;
c) basil oil;
d) davana oil;
e) rosewood oil;
f) fennel oil; and
g) citronella oil;
wherein the mixture of said oils is in the ratio of A:B:C:D:E:F:G;
wherein:
said component A is said lemon oil with a relative ratio of 2 to 6;
said component B is said eucalyptus oil with a relative ratio of 5 to 9;
said component C is said basil oil with a relative ratio of 3 to 6;
said component D is said davana oil with a relative ratio of 1 to 4;
said component E is said rosewood oil with a relative ratio of 1 to 4;
said component F is said fennel oil with a relative ratio of 1; and
said component G is said citronella oil with a relative ratio of 6 to 12.

2. The formulation according to claim 1, wherein
the relative ratio of said component A is 2 to 5;
the relative ratio of said B is 5 to 8;
the relative ratio of said C is 3 to 5;
the relative ratio of said D is 1 to 3;
the relative ratio of said E is 1 to 3;
the relative ratio of said F is 1; and
the relative ratio of said G is 6 to 11.

3. The formulation according to claim 2, wherein
the relative ratio of said A is 2 to 4;
the relative ratio of said B is 5 to 7;
the relative ratio of said C is 3 to 4;
the relative ratio of said D is 1 to 2;
the relative ratio of said E is 1 to 2;
the relative ratio of said F is 1; and
the relative ratio of said G is 6 to 9.

4. A method of treatment of hypoxia and other related disorders comprising administering an effective amount of the formulation according to any one of claims 1 to 3.

5. The method according to claim 4 wherein said hypoxia is selected from the group consisting of: blood hypoxia, cerebral hypoxia, and tissue and cell hypoxia.

6. The formulation according to any one of claims 1 to 3, wherein said formulation further comprises a supplement.

7. The formulation according to any one of claims 1 to 3, wherein said formulation is in a form selected from the group consisting of: spray, patches, cream, ointment, powder, capsules, and liquid.

8. The formulation according to any one of claims 1 to 3, wherein said formulation is in a form suitable for topical use, inhaler use, or oral use upon dilution of said formulation.

9. A cleansing product or skin care product comprising the formulation according to any one of claims 1 to 3.

10. The product according to claim 9, wherein said product is selected from the group consisting of: shampoo, cream, and soap.

11. Use of the formulation according to any one of claims 1 to 3 for the enhancement of human physical strength and athletic performance.

12. A method of treatment of hypoxia and related disorders comprising administering an effective amount of a plant extract formulation, wherein said formulation comprising a mixture of
a) lemon oil;
b) eucalyptus oil;
c) basil oil;
d) davana oil;
e) rosewood oil;
f) fennel oil; and
g) citronella oil;
wherein the mixture of said oils is in the ratio of A:B:C:D:E:F:G;
wherein:
said component A is said lemon oil with a relative ratio of 1 to 4;
said component B is said eucalyptus oil with a relative ratio of 1 to 4;
said component C is said basil oil with a relative ratio of 1 to 4;
said component D is said davana oil with a relative ratio of less than 3;
said component E is said rosewood oil with a relative ratio of 1 to 4;
said component F is said fennel oil with a relative ratio of 1; and
said component G is said citronella oil with a relative ratio of 2 to 5.

13. The method according to claim 12, wherein
the relative ratio of said A is 1 to 3;
the relative ratio of said B is 2 to 4;
the relative ratio of said C is 2 to 4;
the relative ratio of said D is less than 2;
the relative ratio of said E is 1 to 3;
the relative ratio of said F is 1; and
the relative ratio of said G is 2 to 4.

14. The method according to claim 13, wherein
the relative ratio of said A is 1 to 2;
the relative ratio of said B is 3 to 4;
the relative ratio of said C is 2 to 3;
the relative ratio of said D is less than 1;
the relative ratio of said E is 1 to 2;
the relative ratio of said F is 1; and
the relative ratio of said G is 3 to 4.

15. The method according to any one of claims 12 to 14, wherein said hypoxia is high altitude hypoxia.

16. The method according to any one of claims 12 to 14, wherein said formulation further comprises a supplement.

17. The method according to claim 16, wherein
said supplement is corn oil;
the mixture of said oils is in the ratio of A:B:C:D:E:F:G:H; and
said component H is said corn oil with a volume ratio of 40 to 70.

18. A method of treatment of hypoxia and related disorders comprising administering an effective amount of a formulation according to claim 17.

19. The method according to claim 18 wherein said hypoxia is high altitude hypoxia.

20. A method of enhancing human immunity comprising administering an effective amount of a plant extract formulation, wherein said formulation comprises an effective amount of citronella oil.

21. The method according to claim 20 wherein said formulation further comprises lemon oil, eucalyptus oil, basil oil, davana oil, rosewood oil, fennel oil, or any combination thereof.

22. A method of treatment of skin related diseases comprising administering an effective amount of a plant extract formulation, wherein said formulation comprises an effective amount of citronella oil.

23. The method according to claim 22 wherein said formulation further comprises lemon oil, eucalyptus oil, basil oil, davana oil, rosewood oil, fennel oil, or any combination thereof.

24. The method according to claim 22 wherein said skin related disease is selected from a group consisting of: inflammatory skin diseases, viral skin diseases, bacterial skin diseases, fungal skin diseases, and radiation-related skin diseases.

25. The method according to claim 24 wherein said skin related disease is selected from a group consisting of: rashes, acnes, and sunburn.

26. A method of enhancing skin quality comprising administering an effective amount of a plant extract formulation, wherein said formulation comprises an effective amount of citronella oil.

27. The method according to claim 26 wherein said formulation further comprises lemon oil, eucalyptus oil, basil oil, davana oil, rosewood oil, fennel oil, or any combination thereof.

28. The method according to claim 26 wherein said enhancement of skin quality is selected from a group consisting of: restoration of skin flexibility, minimizing of skin pore size, whitening of skin, removal of dark-eye circle, and removal of wrinkles.
